SEQUENCE LISTING (3 sequences)

SEQ ID NO: 1 - DNA, 1295 bp, Pelargonium
gtgacattgacgtgttagaggatatgtcaatgaagttacacattcataattgagcgtgtcattgttgtaattactcttaaaaaaattgtattattgagtccttatgtcgaccatttgttttagtacttgacactacgtatatttaatatttctcaataaaataaatgctatagtataaaattaacgttaaacctttaagtataatataaacaattttgagatttgttaagagcacttacattagaagatctcaccgtttgaaaattaatatatggctccacattttgtaagatccaccaacgccggagaccttaaataaccactgtagaaggtattttgaaagtgtacaattactaaaatgaacatgaaatttaatatatatatatatatatatatatatatatatttagctatattacacagatcattttgacacctggtttgtaatctcagtccaaacctacacattacagtcggcctacctctatcaaggttaacatgcacaacacaatcgacctacgtctctcatggtcgaccttctacactacttacaaacatcgtcaaaggccgacttctattaaattaaagatggtcgacctactattatcgactacccaaatgatcatacagtcaaccttcacatatcaacaacacacaagatgaatggtaactatgtaagataatagaataaaataaaagacaaatgtgagatatttttaactctctcttccttttgcccaaaacggaagtaaatacctcgtttacataagaatgtgcaaacatggttgggaagaagctttcagtccaaagctccgttatgagcaaacaatttctatgtagttgaaggagctttatcctacgtggcatatctggaaggaggcaagaggagggtgtttgatgttccggaacaaggccttagttgaatcaaagtaggaagagagtgagaactaagccaccaatagttttgtcagtcatataatttattaagtgaaagaggagagcagaggccagttgcaatagaaaatgaaataaatgttggccacaacaccaaccggtggtattaggactttcaaacaatactactgcgagtgtgcaaggaaccttcgcctctgactctacttccctaactcctataaataatcggatcccacggaacctccaacatcacaacctcaataatctcaattcatctcctatctacaccttcacagcacagctctttaagcaaccatcatcatcttttgcatattaattctgaggattttctttgagcaaaacaacatcgatcaaaaatg

SEQ ID NO: 2 - DNA, 23 bp, Artificial Sequence
  i 3, 6, 9, 12, 18, 21: PCR primer n=i
ggnytnccnggnttymgnrtngg

SEQ ID NO: 3 - DNA, 23 bp, Artificial Sequence
  i 3, 5, 6, 15, 18: PCR primer n=i
canannckraasmanccnrsytc

(19) United States
(12) Patent Application Publication
(10) Pub. No.: US 2004/0101841 A1
(43) Pub. Date: May 27, 2004

(54) Plant promoter

(76) Inventor: Rajinder S. Ranu, Fort Collins, CO (US)

Correspondence Address: Santangelo Law Offices, Third Floor, 125 South Howes, Fort Collins, CO, 80521, US

(21) Appl. No.: 10/275,802

(22) PCT Filed: May 9, 2001
(86) PCT No.: PCT/US01/15023

(51) Int. Cl. C12Q 1/68; C07H 21/04; A01H 1/00; C12N 15/82

(52) U.S. Cl. 435/6; 536/23.2; 800/283

(57) ABSTRACT

Promoter sequences identified in the genomic clone of PHSacc49 provide technology by which expression of a sense or antisense genes may be driven in transgenic plants. Sense and introduced antisense genes expression can be regulated by the same endogenous promoter to the same extent. Moreover, as a promoter native to geranium, its activity will be influenced by endogenous and exogenous signals in the same fashion and regulation of ethylene levels in plants would represent a condition that is natural to the plant.

This application claims the benefit of U.S. Provisional Patent Application No. 60/203,021, filed May 9, 2000 and U.S. Provisional Patent Application No. 60/239,782, filed Oct. 12, 2000, each hereby incorporated by reference.

### I. TECHNICAL FIELD

Promoter DNA sequences for geranium are identified in the genomic clone of PHSacc49. These promoter sequences may provide a means to regulate the level of transcription of a coding sequence in geraniums and other plants. Specifically, sense and introduced antisense PHSacc49 genes could be driven in transgenic plants.

### II. BACKGROUND

Eukaryotic genes consist of a transcription-translation initiation region, a coding region and a termination region. The transcription initiation region is typically located upstream of the coding region. This initiation region includes a promoter region. The promoter region is responsible for inducing transcription of the coding region and of untranslated sequences responsible for binding of ribosomes and for translation initiation.

The characteristics of the promoter will determine the level and timing of transcription. A promoter typically consists of a “TATA box” and an “upstream activating region.” The TATA box is responsible for marking the initiation of transcription approximately 25 base pairs in the 3′ direction toward the start of the coding region.

Through recombinant techniques, a plant transcription-translation initiation region can be designed to activate a homologous or heterologous (or non-naturally occurring) gene. By manipulation of the promotor the timing and level of expression of transcription can be controlled.

Transcription Elements of ACC Synthase Genes. The transcriptional elements of genomic ACC synthase genes have been identified from variety of plants. In LEACS2 of tomato ACC synthase genes, an AT-rich promoter element exists between two highly G-rich regions at approximately −430 and −500 up stream from the transcription initiation site. A TATA box is located at −32 position. The 5′-flanking region of this gene contains 6 to 10 bp long common sequences with E4 promoter, which is regulated by ethylene (Rottman et al., 1991). And additional regulatory sequences have been identified based on binding sites of conserved DNA binding protein in other organisms. SP I,myc, AP2 and octamer binding sites are contained in LE-ACS2 promoter. In another tomato genomic ACC synthase gene, LE-ACS4, promoter contains TATA-box at position −35 and CAAT box at position −24. LE-ACS4 promoter also contains common sequences with E4, and it includes G-box like sequence, whose core sequence is CACGTG, at position −610 (Lincoln et al., 1993). From these results, these two ACC synthase genes could be regulated by similar inducible elements. In Arabidopsis thaliana, ACS4 promoter contains TATA box at position −29 and CAAT box at position −119 (Able et al., 1995). This gene promoter also includes four putative auxin-responsive elements (Aux RE). These Aux RE sequences at position −404 are common to OS-ACSI from rice, at positions −448 and −359 have high homology with PS-IAA4/5 from Pea, and at position −271 is similar to the soy bean GI-I 3 promoter (Abel et al., 1995). These results suggest that ACS4 gene may, at least in part, be regulated by auxin (Abel et al., 1995). In another ACC synthase gene in Arabidopsis, ACSI, putative CAAT and TATA box were determined. By comparison between ACS I promoter and other ACC synthase promoters in various plant species, ACS I promoter appears to have the highest similarity at positions approximately −500 and −750 to CP-ACC1A promoter of Zucchini, and this region partially overlaps with LE-ACS2 promoter of tomato (Huang et al., 1991; Van Der Stracten et al., 1992). Moreover, ACS1 promoter includes similar sequences to ethylene-responsive promoter and wound inducible promoters. G-box like sequence is also detected at position at −905 (Van Der Straeten et al., 1992). In Nicotiana spp and some other plants, GCC box, a 11 bp sequence, TAAGAGCCGCC, is conserved in the 5′ upstream region of ethylene inducible pathogenesis-related protein gene (Takagi and Shinshi, 1995). This sequence is essential for ethylene responsiveness when incorporated into a heterologous promoter. It is clear that many ACC synthase promoters include regulatory elements which respond differentially to various signals in the plant environment.

### III. DISCLOSURE OF THE INVENTION

The broad object of this invention is to provide a promoter sequence for the regulation of transcription-translation in plant cells. The promoter sequences disclosed may be incorporated into a variety transgenic plant host cells to manipulate the level of expression of proteins.

A significant object of an embodiment of the invention may be to provide for a vector having the promoter invention operably linked to a homologous or heterologous nucleic acid sequence. The heterologous nucleic acid sequence may, as but one example, encode for antisense RNA complementary to at least a portion of the ACC synthase gene from Pelargonium or Rosa as, for example, described in U.S. Pat. No. 5,824,875 or patent applications PCT 97/17644; and U.S. Ser. No. 09/171,482, hereby incorporated by reference.

Another significant object of an embodiment of the invention may be to provide for expression cassettes that have the promoter invention operably linked to a heterologous nucleic acid sequence as, for example, described in the above-mentioned or and patent applications or as may be well known to those skilled in the art.

Another significant object of an embodiment of the invention may be to provide for transgenic plants that have the promoter operably linked to a heterologous nucleic acid sequences as, for example, described in the abovementioned patents or patent applications or as may be well known to those skilled in the art.

Yet another significant object of an embodiment of the invention may be to provide methods of expressing heterologous nucleic acid sequences in plant cells as described, for example, in the above-mentioned patents and patent applications or as may be well known to those skilled in the art. In these methods, a plant cell is transformed with a vector that has the promoter invention operably linked to a heterologous nucleic acid sequence. After transformation with the vector, the plant cell is grown under conditions where the heterologous nucleic acid sequences are expressed.

### IV. BRIEF DESCRIPTION OF THE DRAWINGS

FIG. 1 shows the nucleotide sequences of genomic PHSacc49 clone (gPHSacc49) upstream of the transcription start site.

FIG. 2 shows the primers used to amplify DNA fragments of PHSacc genes by polymerase chain reaction.

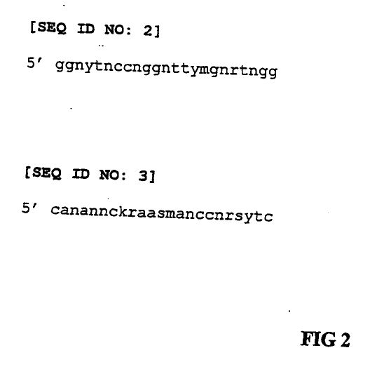

### V. MODE(S) FOR CARRYING OUT THE INVENTION

The invention constitutes promoter sequences for geranium identified in the genomic clone of PHSacc49. This promoter sequence technology may satisfy a long felt need for a native geranium promoter which may regulate 1-aminocyclopropane-1-caroxylate synthase genes from Pelargonium and other plants so that ethylene levels in plants may be controlled. These promoter sequences identified in the genomic clone of PHSacc49 may be the means by which expression of antisense PHSacc49 gene could be driven in transgenic plants. The sense and introduced antisense genes expression would be regulated by the same endogenous promoter to the same extent. Moreover, as a promoter native to geranium, its activity will be influenced by endogenous and exogenous signals in the same fashion and regulation of ethylene levels in plants would represent a condition that is natural to the plant.

Extraction of High-Molecular Weight Genomic DNA. The greenhouse grown Pelargonium x hortorum cv. Sincerity was used for extraction of the genomic DNA. Young healthy leaves were harvested and frozen immediately in liquid nitrogen. Total DNA isolation was performed according to Guillemaut and Marechal-Drouard (1992) with minor modification. Five grams of leaf tissue was ground into a fine powder in liquid nitrogen and transferred into a 50 ml conical tubes containing 25 ml of pre-warmed (65° C.) extraction buffer [100 mM sodium acetate (pH 5.5), 50 mM EDTA (pH 8.0), 500 mM NaCl, 2% soluble PVP 10,000, and 1.4% SDS]. The leaf powder and extraction buffer mixture were incubated at 65° C. (in a water bath) for 1 hour and gently mixed several times during the incubation period. The mixture was centrifuged at 10,000 rpm for 10 min at 25° C. and the supernate transferred to a fresh tube containing 7.5 ml of 5 M potassium acetate (pH 4.8) solution. The mixture was incubated at 0° C. for 30 min and then centrifuged at 10,000 rpm for 10 min at 4° C. DNA in extract was precipitated with isopropanol (0.6 volume). DNA precipitate was recovered by centrifugation at 10,000 rpm for 10 min at 4° C. and the pellet dissolved in 5 ml of H₂O. Any insoluble material was removed by centrifugation at 10,000 rpm for 10 min at 4° C.; the DNA re-precipitated with 0.6 volumes of isopropanol. The precipitate was recovered (by centrifugation at 10,000 rpm for 10 min at 4° C.) and washed twice with 70% ethanol. It was dissolved in 700 μl of TE buffer (10 mM Tris-HCl, 1 mM EDTA, pH 8.0). RNAse (1 μg/ml) was added to the sample to remove RNA by incubation at 37° C. for 1 hour.

Partial Digestion of genomic DNA with Sau3A and Isolation of Size-Fractionated DNA fragments. In order to isolate overlapping DNA fragments, the genomic DNA was digested with Sau3A restriction enzyme as follows. Genomic DNA (210 μg in 150 μl) was dispensed in a Sau3A enzyme reaction buffer (10×containing 200 mM Tris-HCl pH 7.5, 600 mM KCl, and 7.5 mM MgCl₂), and transferred into six pre-chilled sterile microfuge tubes. With the exception of the first tube that had 60 μg of DNA in 30 μl, each of the remaining tubes received 30 μg of DNA in 15 μl. To the first tube 0.703 Units of Sau3A restriction enzyme (USB) was added and mixed well. One half of the first tube reaction mixture was transferred to the second tube, mixed well, and the two-fold serial dilution process was continued through the remaining tubes. The DNA was digested at 37° C. for 30 min and the reaction was stopped by the addition of diethylpyrocarbonate (DEPC) and EDTA to a final concentration of 0.1% and 10 mM, respectively. Samples were incubated at 65° C. for 5 min. The digested DNA samples were extracted with phenol: chloroform, pooled and DNA precipitated with 2 volumes of 95% ethanol (ETOH) in the presence of 200 mM NaCl. It was washed with 70% ethanol and DNA pellet dissolved in 200 μl of TE buffer.

Isolation of DNA fractionated according to size. A sucrose density gradient (10-40%) was prepared [in a buffer containing 10 mM Tris-HCl (pH 8.0), 10 mM NaCl and 1 mM EDTA] in a Beckman SW40 polyallomer centrifuge tube. The partially digested DNA samples were heated at 68° C. for 10 min and then quickly cooled. Aliquots (200 μl) were carefully layered on top of each gradient and the samples were centrifuged at 22,000 rpm for 22 hours at 20° C. in a Beckman SW40 rotor. The gradient tube was punctured from the bottom and about 0.5 ml aliquot fractions were collected. Aliquots (five μl) of each sucrose gradient fraction were removed and applied to a 0.8% agarose gel in TBE buffer. The agarose gel was electrophoresed at 5 V/cm for 2 hours. Gradient fractions containing 9 to 23 kilo base pairs (kbp) DNA fragments were pooled and dialyzed against TE buffer overnight at 4° C. The dialyzed DNA was precipitated with two volumes of 95% ETOH in the presence of 200 mM NaCl and washed twice with 70% ETOH. The resulting DNA sample was dried at room temperature and dissolved in 200 μl of TE buffer.

Treatment of DNA With Calf Intestine Alkaline Phosphatase. Calf intestine alkaline phosphatase (CIP) was used to remove 5′-phosphate from the size fractionated DNA to prevent self-ligation during the ligation reaction. Approximately 8 μg of DNA insert was treated with 5 units of CIP (Boehringer Mannheim) in dephosphorylation buffer [10×containing 0.5 M Tris-HCL (pH 8.5), 1 mM EDTA] in a final volume of 50 μl at 37° C. for 1 hour. After CIP treatment, the enzyme was removed by digestion with proteinase K (100 μg/ml) in the presence of 0.5% SDS at 56° C. for 30 min. The dephosphorylated DNA was purified by phenol/chloroform extraction (Sambrook et al. 1989).

Construction of Genomic Library. Lambda DASH II replacement vector (Stratagene Inc, 1997) was used to construct the genomic library because it allows cloning of large fragments of genomic DNA. Also the Lambda DASH II system takes advantage of spi (sensitive to P2 inhibition) selection. Lambda phages containing active red and gam genes are unable to grow on host strains that contain P2 phage lysogens (Kretz et. a., 1989, Kretz and Short, 1989, and Kretz et al., 1991). Lambda phages without these genes are able to grow on strains lysogenic for P2 such as XL I-Blue MRA(P2), which is a P2 lysogen of XL I-Blue MRA. The red and gam genes are located on the stuffer fragment, therefore, the wild-type Lambda DASH II phage cannot grow on XLI-Blue MRA (P2). When the stuffer fragment is replaced by an insert, the recombinant Lambda DASH II becomes red/gam negative, and the phage is able to grow on the P lysogenic strain, By plating the library on the XL I-Blue MRA (P) strain, only recombinant phages are allowed to grow.

Ligation of Genomic Inserts Into Lambda DASH II Vector. Lambda DASH II/BamHI and Sau3A partial digested genomic DNA share complimentary single-stranded sequence GATC. Ligation of the BamHI predigested Lambda DASH II arms (Stratagene) and size selected Sau3A-cut genomic DNA inserts was achieved by mixing them together at a concentration of DNA that favors the formation of concatenated Lambda DNA. Two μg of size-selected insert and 1 μg of Lambda/BamH1 arms were ligated in the presence of 2 units of T4 ligase and 1 mM ATP in a final volume of 5 μl reaction. The ligation reactions were carried out at 4° C. for 24 hours.

In Vitro Packaging and Titration of the Genomic Library. Gigapack III (Stratagene Inc., 1997) Lambda packaging extracts were used for in vitro packaging of recombinant Lambda. Two μl of the ligation reaction mixture was transferred into 50 μl of packaging extract and incubated at 22° C. for 90 min. SM buffer (500 μl) and 20 μl of chloroform were added into the packaged reaction mixture and mixed on a vortex. After a quick spin at 10,000 rpm (to sediment the debris), the supernate was assayed for phage titer. To determine the phage titer, 10 ml of LB broth (containing 0.2% maltose and 10 mM MgSO₄) was inoculated with a single colony of bacterial strains of XL I-Blue MRA (P). The cells were incubated overnight at 37° C. with shaking. The following day, 500 μl of overnight cell cultures were transferred into 25 ml of LB broth containing 0.2% maltose and 10 mM MgSO₄. The cells were incubated at 37° C. with vigorous shaking until OD₆₀₀ reached approximately 0.8-1.0. Cells were collected by centrifugation at 4,000 rpm at 25° C. for 10 min. The cell pellet was re-suspended in 10 mM MgSO₄ solution to an OD₆₀₀ of approximately 0.5. Two hundred μl of diluted cells were mixed with one μl of serial dilutions (10−², 10−⁴) of the recombinant library of the packaged phages in Falcon 2059 tubes. The mixtures were incubated at 27° C. for 15 min. for phage attachment to the cells. Four ml of pre-warmed (48° C.) top agarose was added to each tube, mixed well, and poured onto 100 mm bottom agar petri dish. The plates were incubated at 37° C. for 5-8 hours until clear plaques were visible.

Isolation of ACC synthase cDNA insert (pPHacc41 and pPHSacc49) for probe preparation. The ACC synthase cDNAs from Pelargonium x hortorum cv. Sincerity cloned in a plasmid pBK-CMV were used as probes for southern hybridization (Fan et. al. 1996). The bacteria transformed by the plasmid was cultured in LB broth containing 50 μg/ml Kanamycin overnight. The plasmid DNA was extracted by Wizard™ plus minipreps DNA purification system (Promega). The plasmid DNA was digested with 10 U of Bam HI and Not I at 37° C. for 3 hours to release the cDNA insert. The digested DNA was electrophoresed in 1.0% agarose gel in TAE (Tris-Acetate-EDTA) buffer at 60 V for 1 hour. The ACC synthase cDNA insert (1945 bp for PHSacc41 and 1878 bp for PHSacc49) in the gel was cut out and extracted from the agarose gel by GenElute™ Minus EtBr Spin Columns (Supelco) and used to prepare [³²P] labeled or digoxigenin-labeled (non-radioactive) probe. Non-radioactive labeling was carried out by, using the Genus I DNA labeling kit (Boeringer Mannheim). The probes were labeled by using random primed method with digoxigenin-11-ddUTP as described in the instructions manual provided by Boeringer Mannheim. Approximately 3 μl of extracted ACC synthase cDNA was denatured with boiling water for 10 minutes in the presence 5 μl of random primers and mixed with 2 μl of dNTP labeling mix and one μl of 2 Units/μl Klenow DNA polymerase, and sterile H₂O was added to a final volume of 20 μl. The reaction mixture was incubated at 37° C. overnight, and 5 μl of 200 mM EDTA was added to stop the reaction. Then one μl of 20 mg/ml glycogen solution, 0.1 volume of LiCl, and 3.0 volumes of 70% cold ethanol were added to precipitate labeled DNA. The mixture was mixed well and incubated at −70° C. It was centrifuged at 10,000 rpm for 5 minutes, and the pellet was washed with 70% of ethanol. After drying, the pellet was dissolved in 50 μl of TE buffer. The Mega-Prime [³²P] labeling system from Amersham Life Sciences was used to prepare radioactive probe according to the manufacturer's protocol.

Screening For Genomic ACC Synthase Gene(s). Approximately one million plaque forming units (pfu) of amplified genomic library were plated for each primary screening at a density of about 50,000 pfu/plate. The plates were incubated at 37° C. for 5-6 hours until clear plaques were visible. The plates were then chilled at 4° C. for at least 1 hour. Hybond-N+ nylon transfer membranes (Amersham) were used to lift the plaques. In the first lift plaques were allowed to remain in contact of the membrane for 2 min and the duplicate lift (second lift) for 4 min. The phage DNA was denatured by placing filters for min in denaturation solution (1.5 M NaCl, 0.5 M NaOH) and neutralized in 1.5M NaCl −0.5M Tris-HCL (pH 8.0); and finally washed in 2×SSC (0.3 M NaCl, 0.03 M sodium citrate) buffer for 2-5 min. The membranes were dried at room temperature for 10 min. The transferred DNA was fixed onto the membranes by UV-cross linking (Strategene UV Cross-Linker), followed by incubation in an oven at 80° C. for 1 hour. The membranes were treated with Rapid-Hyb Buffer (Amersham) at 60° C. for four hours for prehybridization and then hybridization was performed at 60° C. overnight in Rapid-Hyb buffer at a probe concentration of 2×10⁶ counts/min/ml of [³²P]. The membranes were washed in the following manner:

With one liter of 2×SSC containing 0.1% SDS at room temperature for 15 min; the wash was repeated once. Then with one liter of 0.1×SSC and 0.1% SDS at 60° C. for 15 min; the wash was repeated. To detect the radioactive signal, washed membranes were wrapped with Saran wrap and exposed to X-ray film (FUJI-RX) in Kodak X-Omatic cassettes at −80° C. for at least 48 hours. The films were developed. A total of seven putative positive clones were identified. On second screening, only two putative positive clones showed strong positive signal. On tertiary screening all of plaques from the two putative positive clones from second screening again showed strong positive signal and were plaque purified.

Preparing Stocks of Positive Clones. The Plate Lysate Method (Sambrook et al. 1989) was used to prepare the stock from a single plaque of each of the two recombinant lambda phage clones. Approximately 106 bacteriophages were plated on a 150 mm plate and incubated at 37° C. overnight. The phages were harvested by overlaying 5 ml of SM buffer onto the plates. The plates were stored at 4° C. with gently shaking for 2 hours. The phage solution was collected into sterile polypropylene tubes containing 100 μl of chloroform. The stock was stored at 4° C.

Extraction of Recombinant lambda DNA. For large-scale phage DNA isolation, the host cells were infected at high multiplicity. An overnight XL1-Blue P2 cells (1 ml) cultures were inoculated in 500 ml of pre-warmed NZY broth containing 0.2% maltose at 37° C. with vigorous shaking (300 rpm). When OD at 600 nm reached to approximately 0.5, 10¹⁰ pfu of phage stock was added and the incubation continued until lysis occurred. Chloroform (10 ml) was added to the culture, and the incubation continued for another 10 minutes. The lysed culture was treated with DNAase I and RNAase (1 μg/ml) at 37° C. for one hour; then solid NaCl and polyethylene glycol (PEG 8000, ICN) were added to a concentration of 1M and 10%, respectively. The mixture was allowed to sit on ice for 2 hours (to precipitate the bacteriophage particles). The bacteriophage particles in the precipitate were recovered by centrifugation and the pellet re-suspended in 8 ml of SM buffer. It was extracted with an equal volume of chloroform. The supernatant (containing the phage) was transferred to a Beckman SW40 tube and the phage particles were recovered by centrifugation at 25,000 rpm for 2 hours at 4° C. The phage pellet was re-suspended in 300 μl of SM. The phage DNA was isolated by incubation of the phage suspension with 50 μg/ml of proteinase K and 0.5% of SDS at 56° C. for one hour. The samples were extracted with phenol/chloroform and the phage DNA precipitated in the presence of 0.3 M sodium acetate (pH 7.0) and 2 volumes of 100% ethanol. The DNA pellet was recovered and washed twice with 70% ETOH. It was air-dried and the DNA dissolved in 100 μl of TE buffer (Sambrook et. al., 1989).

Southern Transfer. Southern blotting was performed by the Alkaline Transfer Method (Amersham). Approximately 2 μg of recombinant lambda DNA which contains ACC synthase genomic DNA from the clones was digested with 20 unit of the following restriction enzymes, Eco RI, Not I, Sac I, Xba I, or Xho I, in a final volume of 20 μl. The digestion mixture were incubated at 37° C. overnight. The digested DNA mixture were electrophoreses in 1.0% agarose gel in TAE buffer at 20 V for 1 hour. The gel was soaked twice in 0.25 N HCl for 10 minutes and then rinsed in distilled water. The DNA in the gel was denatured by soaking in 0.5 N NaOH solution containing 1.5 M NaCl for 1 hour. The gel was neutralized by soaking in 0.5 M Tris HCl buffer (pH 7.0) containing 1.5 M NaCl for 1 hour. The DNA in the gel was blotted to a Hybond-N+ (Amersham) membrane by capillary transfer in 10×SSC buffer overnight (Sambrook et al., 1989). After Southern transfer, the membrane was briefly washed with 5×SSC for 1 minute, and the DNA was fixed by UV light for 120 seconds. Prehybridization and hybridization were performed as described under Primary Screening of Genomic Library. The membrane was placed in a plastic bag containing 20 ml of standard prehybridization solution and then incubated was incubated at 65° C. for 2 hours. The membrane was transferred to hybridization solution containing 20 ng/ml of the Dig-labeled probe in and incubated at 65° C. overnight. The membrane was washed twice with 2×washing solution, 2×SSC with 0.1% SDS, for 5 minutes. Finally the membrane was washed twice with 0.1×SSC containing 0.1% SDS, at 65° C. for 15 minutes.

Color Development. The membrane was washed with color development buffer 1 (set out below) for 1 minute, and then the membrane was soaked in blocking buffer consisting of development buffer 1 and 2% (w/v) blocking reagent for nucleic acid hybridization for 30 minutes to block the non-specific antibody binding sites on the membrane. The membrane was placed anti-DIG-alkali-phosphatase antibody solution diluted 1:5,000 in Blocking buffer for 30 minutes. The membrane was washed twice with 100 ml of color development buffer 1 and then soaked in 20 ml of color development buffer 2 for 2 minutes. Finally, the cloned genomic DNA (including geranium ACC synthase gene) on the membrane was visualized by soaking in 10 ml of Color Substrate Solution [45 μl of 75 mg/ml of nitroblue] tetrazodium salt (NBT) in 70% (V/V) dimethylformamide and 35 μl of 50 mg/ml 5-bromo-4-chloro-3-indolyl phosphate (X-phosphate) toluidium salt in 100% dimethylformamide in 10 ml of development buffer 2] overnight. The membrane was washed with 50 ml of development buffer 1 for 5 minutes to stop the reaction. These results showed that the two genomic clones were identical and belong to pPHSacc 49 gene. Therefore, only one of the clones was used to determine the DNA sequence.

Sequencing of the Genomic ACC Synthase DNA. The sequencing of ACC synthase genomic DNA was performed by the use of [α³³P] labeled dideoxynucletide terminators and the Thermo Sequenase™ DNA polymerase (Fan et al., 1997). This method has two main features. First, the four [α³³P] dideoxynucleotide (ddNTP) terminators (A, C, G, & T) are used to label the properly terminated DNA chains during the elongation of the DNA chains by DNA polymerase. Second, the thermostability of the DNA polymerase is exploited for use in cycle sequencing (Lee, 1991; Ranu, 1995). As a result, labeling and termination reactions are performed in a single step. A very small amount of a template DNA is required. The labeled DNA chains were resolved by high resolution polyacrylamide gel electrophoresis (PAGE) in the presence of urea and subsequently visualized by autoradiography of the sequencing gel. The master mixture was prepared by mixing 500 ng of template DNA, 10 μl of 0.2 pmol of primer, 2 μl of 10× buffer containing 260 mM Tris-HCl (pH 9.5) and 65 mM MgCl₂, and 2 μl of 4 U/μl Thermo-Sequenase DNA polymerase, and the total volume was adjusted to 20 μl with sterile H₂O. For each ddNTP (G, A, T, & C) terminator mixture, 2 μl of dNTPS mixture and 0.5 μl of one [α³³P] ddNTP were prepared. Then 4.5 μl of master mixture was added to each ddNTP termination mixture, and it was mixed well. One drop of mineral oil was overlaid on each tube containing reaction mixture. After initial denaturation at 94° C. for 3 minutes, 50 cycles of thermocycling were performed at 94° C. for 30 seconds, at 4° C. lower than the Tm of the primer's for annealing for 30 seconds, and at 72° C. for 60 seconds for extension. At the end, 4 μl of stop solution 95% formamide, 20 mM EDTA, 0.05% bromophenol blue, 0.05% xylene cyanol FF) was added to each reaction mixture. The mixture was heated at 95° C. for 5 minutes and then cooled on ice. The labeled DNA were electrophoresed in high resolution PAGE in the presence of urea for 2, 5, or 9 hours. The DNA in the gel was soaked in solution containing 10% methanol and 10% acetic acid to remove urea. The gel was transferred to a filter paper and dried. An autoradiograph was prepared by exposing the gel to X-ray film (Kodak, BioMax film) at room temperature for 16-24 hours. The autoradiograph was developed, and the sequence of the DNA was read. Usually, 400-500 bases of sequence is read from one sequencing reaction (Ranu, 1995).

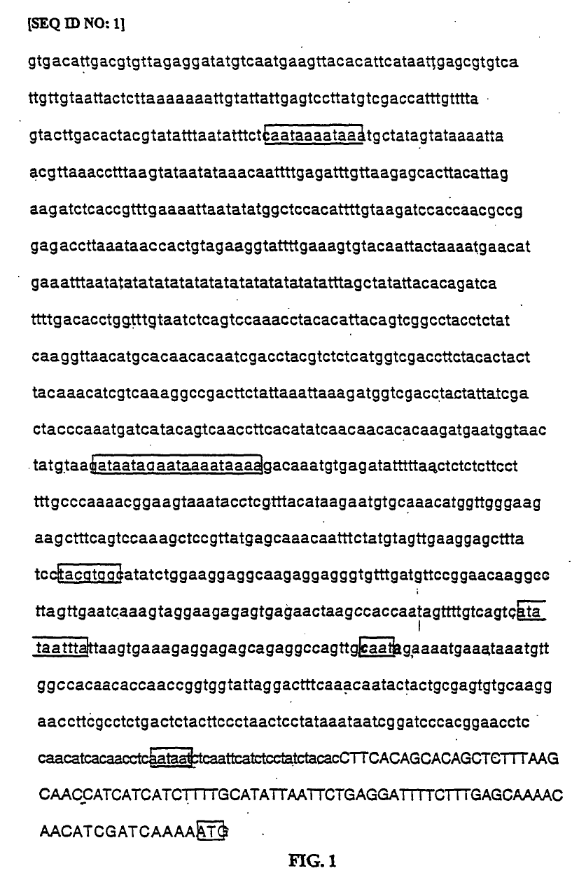

Determination of gPHSacc49 Transcription Initiation Site and Promoter elements. DNA sequence data of gPHSacc49 clone confirmed that it contains sequence homologous to sequences obtained from cDNA of PHSacc49. The putative transcription initiation site has been determined by comparing sequences with PHSacc49 cDNA. These results show that the 5′-untranslated region of the gPHSacc49 is 89 nucleotides long and without any intron in the 5′-untranslated region (FIG. 1). The sequence at position −29 to −24, AATAAT, qualifies as a TATA box (Breathnanch and Chambon, 1981). And putative CAAT box sequence is present further upstream at position −129 to −126 (FIG. 1). There are two AT-rich regions at position −170 and −211, and two GC-rich regions are located at position −146 and −193 (FIG. 1). Detection of regulatory sequences which have been identified as binding sites for conserved DNA binding proteins in other organisms show that the gPHSacc49 promoter contains matching G-box. The G-box like sequence located at −167 is similar to the G-box present in many light-, wound-, and ABA-regulated plant promoters (Guiltinan et al., 1990). Some similarities to other ethylene regulated promoters, E4 and E8 promoters in tomato, (Lincoln and Fischer 1988, and Deikman and Fischer, 1988) are also found in the 5′-flanking region of gPHSacc49. The gPHSacc49 promoter site has 52% overall DNA sequence identity with E4 promoter site and also has 53% overall DNA sequence identity with E8 promoter site, and the greatest sequence matching regions were found in this region. Two sequences of 11 bp and one sequence of 19 bp 1 regions are conserved in gPHSacc49 and E8 promoter sites (Lincoln and Fischer 1988, and Deikman and Fischer, 1988). The elements identified in sequence upstream of the transcription initiation of gPHSacc 49 are consistent with promoter elements found in eukaryotic/plant promoters; some of these elements may represent ethylene regulatory elements.

Mediums, Buffers and Solutions.

LB Broth:

10 g NaCl

10 g Tryptone

5 g Yeast Extract

Dissolved in 800 n-d dH²O

Adjusted pH to 7.0 with 5 N NaOH

Added dH₂O to a final vol. of 1,000 ml

Autoclaved

LB Plates:

15 g agar in 1,000 ml of LB broth

Autoclaved and poured into sterilized petri dishes

NZY Broth:

5 g NaCl

2 g MgSO-7H₂O

5 g Yeast Extract

10 g NZ Amine (casein hydrolysate)

Dissolved in dH₂O

Adjusted pH to 7.5 with 5 N NaOH

Added dH₂O to a final volume of 1,000 ml

NZY Plates:

15 g agar in 1,000 ml of NZY broth

Autoclaved and poured into sterilized petri dishes

NZY Top Agar:

0.7% agar in NZY broth

TE Buffer:

| | | |
|---|---|---|
| 10 | mM | Tris-HCI (pH 7.5) |
| 1 | mm | EDTA |

TAE Buffer:

| | | |
|---|---|---|
| 40 | mM | Tris-acetate |
| 1 | mM | EDTA |

SM Buffer:

| | | |
|---|---|---|
| 5.8 | g | NaCl |
| 2.0 | g | MgS04 |
| 50.0 | ml | 1 M Tris-HCI (pH 7.5) |
| 5.0 | ml | 2% gelatin |

Added dH20 to a final volume of 1,000 ml

20×SSC Buffer:

| | | |
|---|---|---|
| 3 | M | NaCI |
| 300 | mM | sodium citrate |

Adjusted pH to 7.5 with 5 N NaOH
Added dH₂O to a final volume of 1,000 ml

Standard Prehybridization Solution:

| 5x | SSC |
|---|---|
| 1.0% | Blocking reagent for nucleic acid hybridization |
| 0.1% | N-lauroylsarcosine |
| 0.02% | SDS |

Development Buffer 1:

100 mM Tris-HCl (pH 7.5)

150 mM NaCl

Adjusted pH to 7.5 with 5 N HCl

Development Buffer 2:

100 mm Tris-HCl (pH 7.5)

100 mm NaCl

50 mM MgCl,

Adjusted pH to 7.5 with 5 N HCl

Deposits:

The following illustrative plasmid encoding the geranium ACC synthase promoter were deposited at the American Type Culture Collection, Rockville, Md., on May 7, 2001prior to the filing date of this patent application under the requirements of the Budapest Treaty. These deposits were granted the following accession numbers and are hereby incorporated by reference:

1. gPHSacc49 clone comprising SEQ ID NO:1—accession number ______.

As can be easily understood from the foregoing, the basic concepts of the present invention may be embodied in a variety of ways. It involves both gene promoter DNA sequences as well as techniques of regulating genes in plants. In this application, the techniques for the regulation of genes are disclosed as part of the result shown to be achieved by the use of the DNA promoter sequences as described, or portions thereof, and as steps which are inherent to their utilization. They are simply the natural result of utilizing the DNA promoter sequences as intended and described. In addition, while DNA promoter sequences are disclosed, it should be understood that these not only accomplish certain methods but also can be varied in a number of ways. Importantly, as to all of the foregoing, all of these facets should be understood to be encompassed by this disclosure.

The discussion included in this PCT application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims which will be included in a full patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for the full patent application. It should be understood that such language changes and broad claiming will be accomplished when the applicant later (filed by the required deadline) seeks a patent filing based on this provisional filing. The subsequently filed, full patent application will seek examination of as broad a base of claims as deemed within the applicant's right and will be designed to yield a patent covering numerous aspects of the invention both independently and as an overall system. Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “primer” should be understood to encompass disclosure of the act of “priming”—whether explicitly discussed or not—and, conversely, were there only disclosure of the act of “priming”, such a disclosure should be understood to encompass disclosure of a “primer” and even a means for “priming” Such changes and alternative terms are to be understood to be explicitly included in the description.

All references in the disclosure or listed in the list of References attached are hereby incorporated by reference; however, to the extent statements might be considered inconsistent with the patenting of this/these invention(s) such statements are expressly not to be considered as made by the applicant(s).

Thus, the applicant(s) should be understood to claim at least: i) a plant promoter or primer DNA sequences as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, and ix) methods and apparatuses substantially as described hereinbefore and with reference to any of the accompanying examples, and x) the various combinations and permutations of each of the elements disclosed.

In addition, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible in countries such as Australia and the like.

The claims set forth in this specification by are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extention thereon.

We claim:

1. An isolated polynucleotide comprising the nucleotide sequence of SEQ ID NO: 1, or degenerate variants of SEQ ID NO: 1.

2. An isolated polynucleotide comprising the complement nucleotide sequence of SEQ ID NO: 1, or degenerate variants of SEQ ID NO: 1.

3. An isolated polynucleotide as described in claim 1, comprising a fragment of the nucleotide sequence of SEQ ID NO: 1, or degenerate variants of SEQ ID NO: 1.

4. An isolated polynucleotide as described in claim 2, comprising a fragment of the complement nucleotide sequence of SEQ ID NO 1, or degenerate variants of SEQ ID NO: 1.

5. An isolated polynucleotide comprising a nucleotide sequence at least 50% identical to SEQ ID NO: 1, or complement thereof.

6. A DNA the sequence of which comprises SEQ ID NO: 1, operably linked to a heterologous coding sequence.

7. An isolated polynucleotide comprising a nucleotide sequence, or complement thereof, defined by polymerase chain reaction using a primer pair set forth in SEQ ID NO: 2 and SEQ ID NO: 3.

8. An isolated polynucleotide as described in claim 8, comprising a fragment of said nucleotide sequence defined by polymerase chain reaction using said primer pair set forth in SEQ ID NO: 2 and SEQ ID NO: 3, or complement thereof.

9. A single-stranded nucleic acid comprising a nucleotide sequence that hybridizes under stringent conditions to said polynucleotide having the sequence of SEQ ID NO: 1, or complement thereof.

10. The isolated polynucleotide described in claim number 1, wherein said hybridization probe is a fragment of SEQ ID NO: 1 at least 20 nucleotides in length.

11. An isolated polynucleotide comprising a nucleotide sequence that encodes an ACC synthase gene promoter having the nucleotide sequence between about nucleotide 1 and about nucleotide 1203 set forth in SEQ ID NO: 1, or degenerate variants thereof.
